(19) United States
(12) Patent Application Publication
(10) Pub. No.: US 2010/0297778 A1
(43) Pub. Date: Nov. 25, 2010

(54) Conjugate Having Cleavable Linking Agent

(75) Inventors: John G. Konrath, Lake Villa, IL (US); Jeffrey A. Moore, Gurnee, IL (US); Richard J. Himmelsbach, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/468,939

(22) Filed: May 20, 2009

(51) Int. Cl. G01N 33/566; C07K 16/18

(52) U.S. Cl. 436/501; 530/391.3

(57) ABSTRACT

A method and reagent that can be used to eliminate the signal caused by non-specific binding of a labeled conjugate, e.g., a specific binding member attached to a label, to a solid phase, e.g., a magnetic microparticle. The method and the reagent involve the use of a cleavable linking agent to link the label to the specific binding member that specifically binds to the analyte. The use of a cleavable linking agent would allow the release of the label from the specific binding member from the complex comprising the magnetic microparticle, analyte, and labeled conjugate into solution. After the release of the label, the magnetic microparticles having any label non-specifically bound thereto, are removed from the reaction mixture. Only the label, e.g., acridinium, from the labeled conjugate would remain in the elution well. The conjugate that is non-specifically bound through interaction between the label and the solid phase, e.g., a magnetic particle, would remain bound to the solid phase, and would subsequently be removed from the elution well when the solid phase is removed from the elution well and transferred to another well the introduction of additional reagent(s).

### BACKGROUND OF THE INVENTION

1. Field of the Invention

This invention relates to reagents, more particularly, reagents for carrying out immunoassays.

2. Discussion of the Art

The KingFisher™ magnetic particle processor, commercially available from Thermo Fisher Scientific, Inc., Waltham, Mass., is designed to transfer coated magnetic particles between wells containing reagents in order to perform various biochemical processes. Movable magnetic rods are used to capture, transfer, and release the magnetic particles. The KingFisher™ magnetic particle processor has many programmable options, such as, for example, parameters of the magnetic particles, parameters relating to the release of the magnetic particles, incubation times, agitation of liquids in the wells, and sequence of usage of the wells.

The KingFisher™ magnetic particle processor can be used to perform immunoassays. In one embodiment of an immunoassay, a plastic strip holding five adjacent 1 mL wells can be used. The wells contain reagents used in the immunoassay. Magnetic particles are transferred between the wells by means of a movable magnet.

The KingFisher™ magnetic particle processor, used in conjunction with an immunoassay procedure, can increase the sensitivity of the immunoassay, as compared with currently available instruments and analyzers for carrying out immunoassays.

The primary mechanism underlying the improvement in detection of analytes is the use of a larger volume of sample that is used in commercially available immunoassay analyzers. A larger volume of sample contains a larger quantity of analyte. The KingFisher™ magnetic particle processor permits capture of this larger amount of analyte and subsequent quantification thereof.

The process carried out by the KingFisher™ magnetic particle processor improves detection of the analyte through reduction in the background signal resulting from non-specific binding of materials, other than the analyte, to the interior surface of a well. For example, certain conjugates having certain labels, e.g., an acridinium label, will bind to the interior surface of the well. The higher the concentration of conjugate having acridinium label in the reaction mixture, or the higher the concentration of acridinium on the conjugate, the more non-specifically bound conjugate will bind to the interior surface of the well.

Because the KingFisher™ magnetic particle processor moves the complex comprising the magnetic microparticle and the labeled conjugate to another well before the trigger reagent is introduced, and the reading carried out, the non-specifically bound conjugate is left behind, i.e., bound to the interior surface of the well, and, accordingly, will not contribute to the signal read. Non-specifically bound label increases the signal from the sample that is not associated with the analyte, i.e., the background signal. Elimination of the signal resulting from non-specific binding that is not associated with the analyte improves the sensitivity of the immunoassay.

In addition to the type of non-specific binding previously mentioned, there is a second type of non-specific binding. In this second type of non-specific binding, the conjugate containing the label, e.g., an acridinium label, non-specifically binds to the magnetic microparticle. This second type of non-specific binding results in an increase in noise and a reduction in the signal-to-noise ratio. FIGS. 1A, 1B, 1C, 1D, 1E, and 1F illustrate the steps of an immunoassay in which a conventional linkage between the specific binding member and the label in one of the conjugates in an immunoassay is utilized. In FIGS. 1A, 1B, 1C, 1D, 1E, and 1F, there are five rows of containers, i.e., tubes, with five containers, i.e., tubes, in each row. The tubes in which operations for a given process step are being carried out are designated by hatch lines. Below the array of 25 tubes (5 rows×5 tubes/row) are schematic representations of (a) a first conjugate, (b) a sample, and (c) a second conjugate undergoing given operations for a given process step.

FIG. 1A shows tubes 10a, 10b, 10c, 10d, and 10e in row 1 at the starting point of the immunoassay. The tubes in rows 2, 3, 4, and 5 are identical to those in row 1. The magnetic microparticle is designated by the reference numeral 20. The specific binding member attached to the magnetic microparticle is designated by the reference numeral 22. The conjugate containing the magnetic microparticle 20 and the specific binding member 22 attached to the magnetic microparticle is referred to as the first conjugate. The analyte in the sample is represented by the reference numeral 24. The specific binding member attached to the label is designated by the reference numeral 26. The label itself is designated by the reference numeral 28. The conjugate containing the specific binding member 26 and the label 28 is referred to as the second conjugate. In FIGS. 1A, 1B, 1C, 1D, 1E, and 1F, in the first conjugate, the specific binding member 22 is covalently bonded to the magnetic microparticle 20. However, it is not required that the specific binding member 22 be covalently bonded to the magnetic microparticle 20. In an alternative embodiment, the specific binding member 22 can be attached to the magnetic microparticle 20 by means of van der Waals force.

As shown in FIG. 1A, at the beginning of the immunoassay, the first conjugate is introduced into the tube(s) 10a, the sample is introduced into the tube(s) 10b, and the second conjugate is introduced into the tube(s) 10c. FIG. 1B shows that the first conjugate is being mixed with the sample in the tube(s) 10b. The specific binding member 22 of the first conjugate binds to the analyte 24 in the sample. FIG. 1C shows that the reaction product in the tube(s) 10b in FIG. 1B has been transferred to the tube(s) 10c containing the second conjugate, whereupon the specific binding member 26 of the second conjugate binds to the analyte 24 that is specifically bound to the specific binding member 22 of the first conjugate. In FIG. 1D, the complex formed in the reaction shown in FIG. 1C is washed in the tube(s) 10d, in order to remove unbound second conjugate. FIG. 1E shows the effect of the pre-trigger solution, whereby the magnetic microparticle 20 is detached from the specific binding member 22, and the analyte 24 is detached from the first specific binding member 22 and the second specific binding member 26. These activities take place in the tube(s) 10e. In FIG. 1F, the magnetic microparticle 20 is removed from the reaction mixture, whereupon the signal can be measured in order to determine the concentration of the analyte 24 in the sample. In the step shown in FIG. 1F, the non-specific binding of the label 28 in the tube(s) 10e results in the non-specifically bound label 28 becoming part of the reaction mixture that will be used for quantifying the concentration of the analyte 24 in the sample, thereby leading to an inaccurate result. The first conjugate has been removed to the tube(s) 10d. Therefore, it would be desirable to develop a method for removing the non-specifically bound label from the reaction mixture.

### SUMMARY OF THE INVENTION

The invention described herein involves a method and conjugate that can be used to eliminate the signal caused by non-specific binding of the conjugate, e.g., a specific binding member attached to a label, to a solid phase, e.g., a magnetic microparticle. The method and the conjugate involve the use of a cleavable linking agent for linking the label to the specific binding member that specifically binds to the analyte. The use of a cleavable linking agent allows the release of the label from the specific binding member from the complex comprising the magnetic microparticle, the analyte, and the conjugate into solution. After the release of the label, the magnetic microparticles having any label non-specifically bound thereto are removed from the reaction mixture. Only the label, e.g., acridinium, from the conjugate would remain in the elution well.

Any conjugate that is non-specifically bound through interaction between the label and the solid phase, e.g., a magnetic particle, would remain bound to the solid phase, and would subsequently be removed from the elution well when the solid phase is removed from the elution well and transferred to another well before the introduction of additional reagent(s), e.g., a trigger reagent.

If the conjugate is non-specifically bound through interaction of the label and the solid phase, the cleavage of the link between the label and the specific binding member (e.g., antibody) would only release the specific binding member (e.g., antibody). The label that is released from the specific binding member would remain bound to the solid phase.

In the immunoassay described herein, after the complex comprising the magnetic microparticles, the analyte, and the conjugate is formed, the cleavable linking agent is cleaved, the label from the complex is released, the magnetic microparticles are removed from the reaction mixture, the label is triggered, and then the signal is measured. In a sandwich immunoassay format, the immunoassay comprises the steps of:
(a) providing a biological sample suspected of containing an analyte;
(b) providing a first conjugate comprising a solid phase material attached to a specific binding member specific for the analyte;
(c) providing a second conjugate comprising a specific binding member specific for the analyte, a label, and a cleavable linking agent, wherein the specific binding member specific for the analyte and the label are joined by the cleavable linking agent;
(d) mixing (a) the biological sample, (b) the first conjugate, and (c) the second conjugate in a container to form a reaction mixture;
(e) cleaving the label from the second conjugate;
(f) removing the label non-specifically bound to the solid phase material;
(g) measuring the signal generated by the label; and
(h) determining the concentration of analyte in the sample.

In a competitive immunoassay format, the immunoassay comprises the steps of:
(a) providing a biological sample suspected of containing an analyte;
(b) providing a first conjugate comprising a solid phase material attached to a specific binding member specific for the analyte;
(c) providing a second conjugate comprising the analyte, a label, and a cleavable linking agent, wherein the analyte and the label are joined by the cleavable linking agent;
(d) mixing (a) the biological sample, (b) the first conjugate, and (c) the second conjugate in a container to form a reaction mixture;
(e) cleaving the label from the conjugate;
(f) removing the label non-specifically bound to the solid phase material;
(g) measuring the signal generated by the label; and
(h) determining the concentration of analyte in the sample.

The invention also provides a kit for carrying out a competitive immunoassay and a kit for carrying out a sandwich immunoassay.

### BRIEF DESCRIPTION OF THE DRAWINGS

FIGS. 1A, 1B, 1C, 1D, and 1E are a series of schematic diagrams illustrating the procedure of inverse magnetic particle processing utilized by a KingFisher™ magnetic particle processor. In these figures a conventional linkage between the specific binding member and the label in one of the conjugates is utilized in an immunoassay. In these figures specific binding members are shown as being covalently bonded to microparticles. Although not shown in these figures, specific binding members can be attached to microparticles by van der Waals force.

FIG. 2 is a perspective view of a KingFisher™ mL magnetic particle processor.

FIG. 3 is a front view in elevation illustrating a KingFisher™ mL magnetic particle processor suitable for carrying out the procedure of inverse magnetic particle processing to prepare a sample for an immunoassay.

FIG. 4 is a front view in elevation illustrating a KingFisher™ magnetic particle processor suitable for carrying out the procedure of inverse magnetic particle processing to prepare a sample for an immunoassay. This processor utilizes micro-well plates having 96 micro-wells per micro-well plate.

FIG. 5 is a top view of a micro-well plate suitable for carrying out the procedure of inverse magnetic particle processing to prepare a sample for an immunoassay. In FIG. 5, two well strips are removed from the micro-well plate. One of the removed well strips can be seen in a top view format. The other of the removed well strips can be seen in a side elevational view format.

FIG. 6 is a side view in elevation of a tip comb suitable for use with a KingFisher™ magnetic particle processor.

FIGS. 7A, 7B, 7C, 7D, 7E, and 7F are a series of schematic diagrams illustrating the procedure of inverse magnetic particle processing utilized by a KingFisher™ mL magnetic particle processor.

FIGS. 8A, 8B, 8C, 8D, 8E, and 8F are a series of schematic diagrams illustrating the procedure of inverse magnetic particle processing utilized by a KingFisher™ magnetic particle processor. In these figures, a cleavable linking agent between the specific binding member and the label in one of the conjugates is utilized in an immunoassay. In these figures specific binding members are shown as being covalently bonded to microparticles. Although not shown in these figures, specific binding members can be attached to microparticles by van der Waals force.

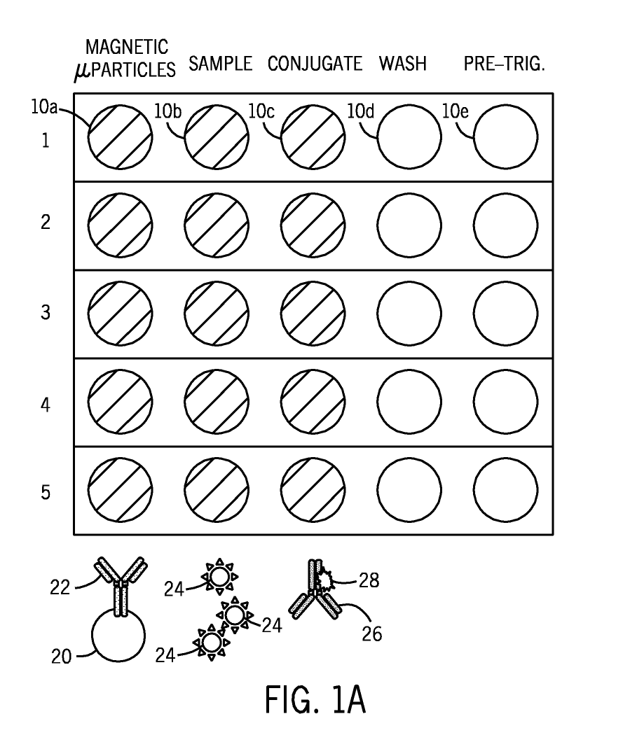

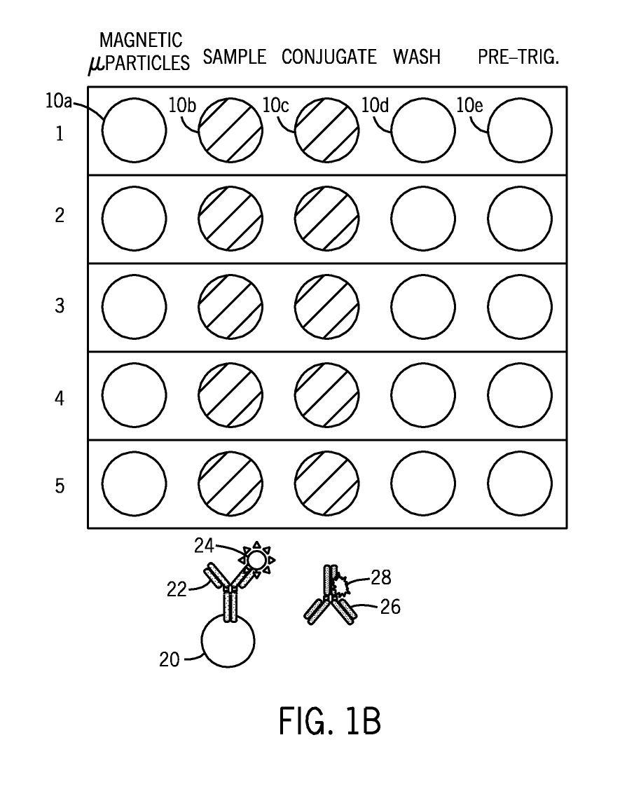

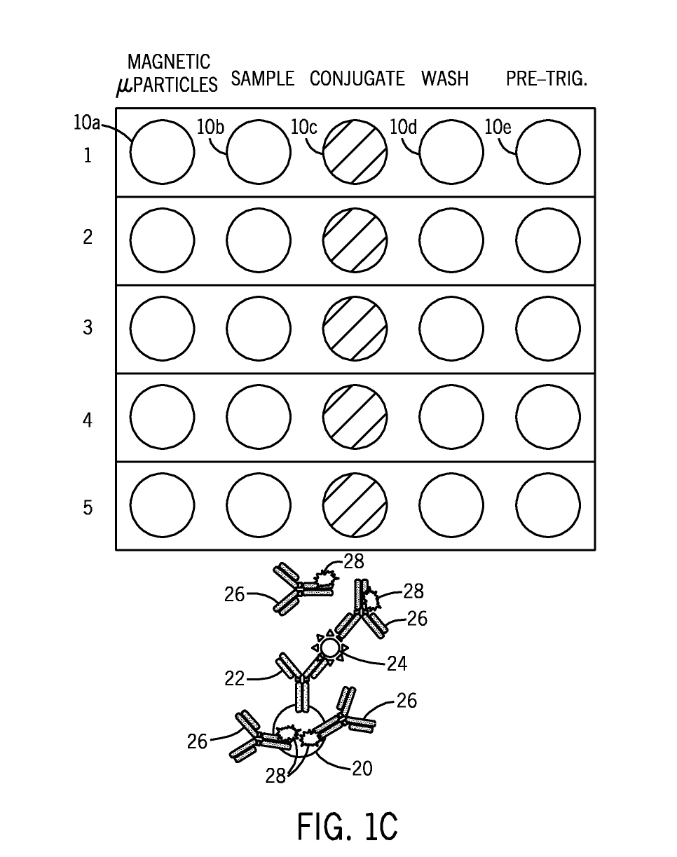

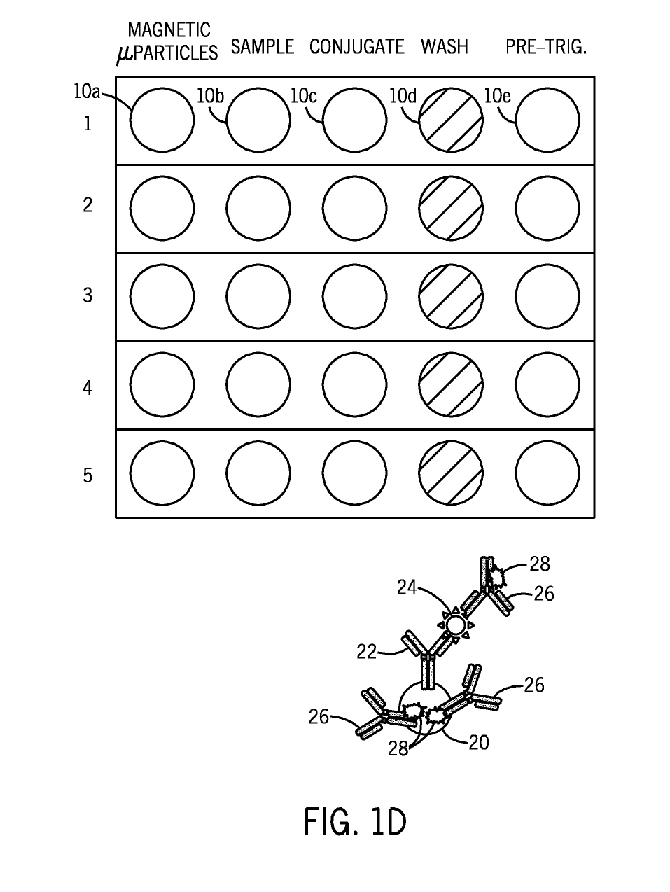

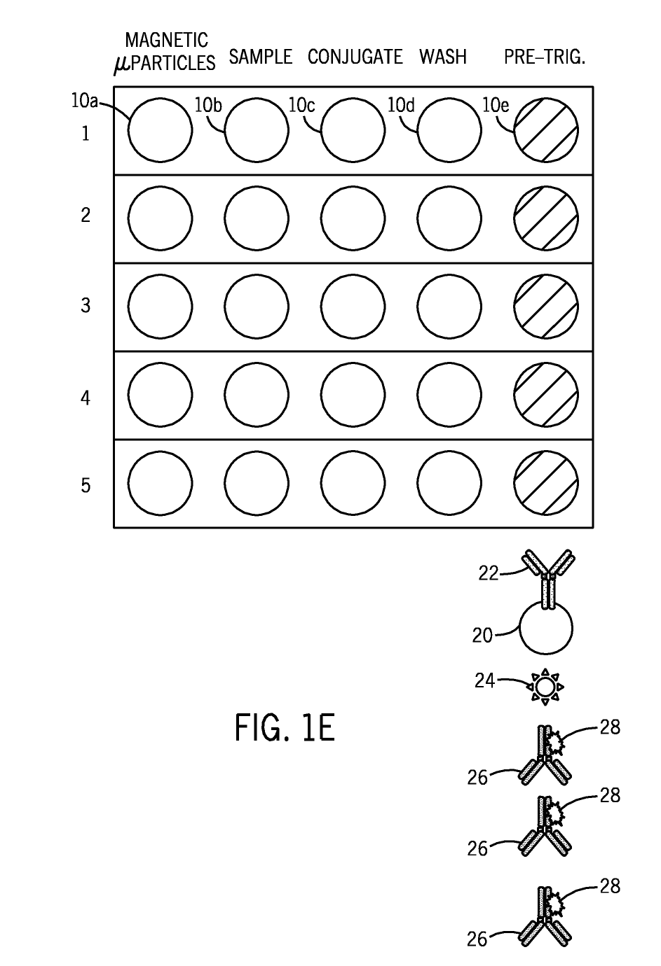

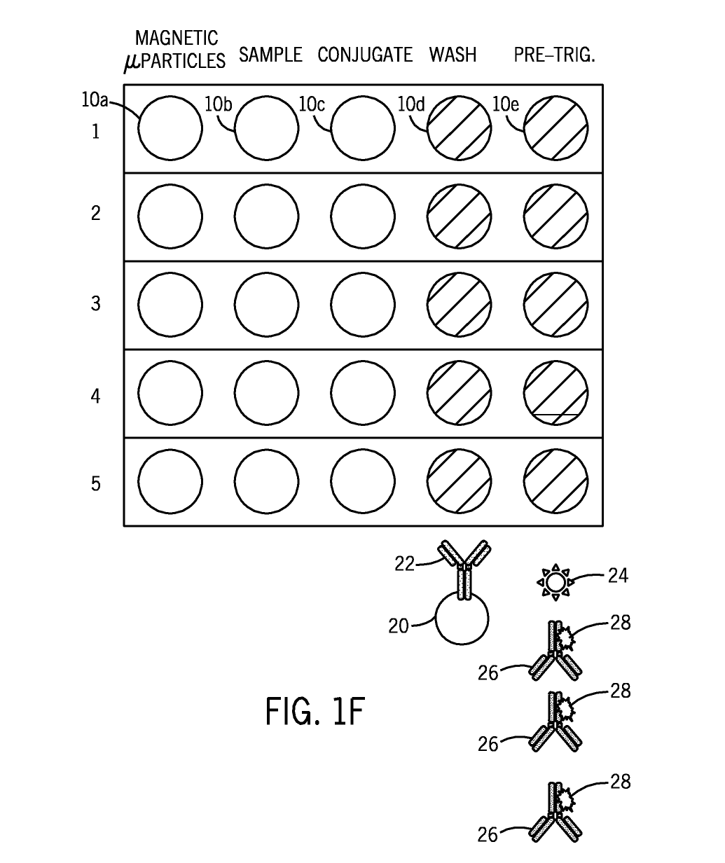

### DETAILED DESCRIPTION

As used herein, the term “container” is intended to include both tubes and wells. The term “well” includes micro-wells and wells having greater volume than a micro-well. The KingFisher™ magnetic particle processor uses micro-wells. The KingFisher™ mL magnetic particle processor uses tubes. The principle of the method and conjugate described herein is the same regardless of whether micro-wells, wells, or tubes are used to perform the immunoassays described herein.

As used herein, the expressions “label”, “label group”, and the like mean a group attached to a specific binding member, e.g., an antibody or an antigen, to render the reaction between the specific binding member and its complementary binding member detectable. Representative examples of labels include enzymes, radioactive labels, fluorescein, and chemicals that produce light. A label is any substance that can be attached to an immunoreactant and that is capable of producing a signal that is detectable by visual or instrumental means. Various labels suitable for use in this invention include catalysts, enzymes, liposomes, and other vesicles containing signal producing substances such as chromogens, catalysts, fluorescent compounds, chemiluminescent compounds, enzymes, and the like. A number of enzymes suitable for use as labels are disclosed in U.S. Pat. No. 4,275,149, incorporated herein by reference. Such enzymes include glucosidases, galactosidases, phosphatases and peroxidases, such as alkaline phosphatase and horseradish peroxidase, which are used in conjunction with enzyme substrates, such as fluorescein di(galactopyranoside), nitro blue tetrazolium, 3,5′,5,5′-tetranitrobenzidine, 4-methoxy-1-naphthol, 4-chloro-1-naphthol, 4-methylumbelliferyl phosphate, 5-bromo-4-chloro-3-indolyl phosphate, chemiluminescent enzyme substrates, such as the dioxetanes described in WO 88100694 and EP 0-254-051-A2, and derivatives and analogues thereof. Preferably, the label is an enzyme and most preferably the enzyme is alkaline phosphatase.

As used herein, the expression “test sample”, the expression “biological sample”, and the term “sample” refer to a material suspected of containing an analyte. The test sample can be used directly as obtained from the source or following a pretreatment to modify the character of the sample. The test sample can be derived from any biological source, such as a physiological fluid, such as, for example, blood, saliva, ocular lens fluid, cerebral spinal fluid, sweat, urine, milk, ascites fluid, synovial fluid, peritoneal fluid, amniotic fluid, and the like. The test sample can be pretreated prior to use, such as preparing plasma from blood, diluting viscous fluids, and the like. Methods of treatment can involve filtration, distillation, extraction, concentration, inactivation of interfering components, the addition of reagents, and the like. Other liquid samples besides physiological fluids can be used, such as water, food products, and the like, for the performance of environmental or food production assays. In addition, a solid material suspected of containing the analyte can be used as the test simple. In some instances it may be beneficial to modify a solid test sample to form a liquid medium or to release the analyte.

As used herein, the expression “specific binding member” means a member of a specific binding pair, i.e., two different molecules where one of the molecules through chemical or physical means specifically binds to the second molecule. An example of such specific binding members of a specific binding pair is an antigen and an antibody that specifically binds to that antigen. Another example of such binding members of a specific binding pair is a first antibody and a second antibody that specifically binds to the first antibody.

As used herein, the term “conjugate” means a specific binding member, e.g., an antigen or an antibody, coupled to a detectable moiety, e.g., a chemiluminescent moiety. The term “conjugate” also means a specific binding member, e.g., an antigen or an antibody, coupled to a solid phase, e.g., a magnetic microparticle.

As used herein, the expression “cleavable linking agent” means an entity that covalently couples a specific binding member to a label, which entity can be cleaved by means of a change in the pH level of less than about 6 or greater than about 8 or by means of a chemical reaction with a chemical entity, such as, for example, a thiol, a periodate, a hydroxylamine.

As used herein, the expressions “solid phase”, “solid phase material”, and the like, mean any material that is insoluble, or can be made insoluble by a subsequent reaction. Representative examples of solid phase material include polymeric or glass beads, microparticles, tubes, sheets, plates, slides, wells, tapes, test tubes, or the like.

As used herein, the term “analyte” means the compound to be detected or measured. The analyte has at least one epitope or binding site.

As used herein, the expression “monoclonal antibodies” means antibodies that are identical because they were produced by one type of immune cell and are all clones of a single parent cell.

As used herein, the term “binding affinity of an antibody” means the strength of the interaction between a single antigen-binding site on an antibody and its specific antigen epitope. The higher the affinity, the tighter the association between antigen and antibody, and the more likely the antigen is to remain in the binding site. The affinity constant is the ratio between the rate constants for binding and dissociation of antibody and antigen. Typical affinities for IgG antibodies are 10⁵ to 10⁹ L/mole.

As used herein, the expression “normal human plasma” means human plasma that is free of the analyte of interest or other known abnormality or pathology.

As used herein, the term “pre-activated” means reacting 1-ethyl-3-(3-dimethylaminopropyl)carbodi-imide hydrochloride (hereinafter “EDAC”) and N-hydroxysulfosuccinimide (hereinafter “sulfo-NHS”) with the carboxyl groups on microparticles to provide semi-stable NHS esters that will react with NH₂ groups on monoclonal antibodies to form stable amide bonds that couple the antibodies to the microparticles.

As used herein, the term “magnetic microparticles” means paramagnetic microparticles. Paramagnetic microparticles are attracted to magnetic fields, hence have a relative magnetic permeability greater than one. However, unlike ferromagnets, which are also attracted to magnetic fields, paramagnetic materials do not retain any magnetization in the absence of an externally applied magnetic field.

As used herein, the symbol “(s)” following the name of an item indicates that one or more of the subject items is intended, depending upon the context. As used herein, the symbol “S/N” means signal to noise ratio.

As used herein, the term “immunoassay” means a special class of assay or test that is performed in a container, e.g., a test tube, a well, a micro-well, which assay or test uses a reaction between and antibody and an antigen to determine whether a patient has been exposed to the antigen or has an antibody to the antigen. An immunoassay can be a heterogeneous immunoassay or a homogeneous immunoassay. The method described herein is primarily concerned with the heterogeneous immunoassay.

Heterogeneous immunoassays can be performed in a competitive immunoassay format or in a sandwich immunoassay format. In the competitive immunoassay format, a solid phase material is attached to a specific binding member specific for the analyte. The sample, which is suspected of containing the analyte, e.g., an antigen, is mixed with (a) the solid phase material attached to the specific binding member specific for the analyte and (b) a conjugate comprising the analyte attached to a detectable moiety. The amount of detectable moiety that binds to the solid phase material can be detected, measured, and correlated to the amount of analyte, e.g., antigen, present in the test sample. The analyte can also be an antibody, rather than an antigen. Examples of solid phase materials include beads, particles, microparticles, and the like.

The present invention is concerned primarily with the sandwich immunoassay format. However, other immunoassay formats, such as, for example, competitive assay formats, can be used. In the sandwich assay immunoassay format, a solid phase, e.g., a microparticle, is coated with antibodies. The antibody on the solid phase is known as the capture antibody. The assay is intended to detect and measure antigens in the sample. A second antibody is labeled with an appropriate label, e.g., acridinium. The second antibody is not attached to a solid phase. The second antibody is known as the detection antibody. The antibody and antigen attach in the following order to form a complex: antibody on solid phase-antigen-antibody having a label. Then the solid phase is removed from the complex. The antibody-antigen-antibody sandwich enables measurement of the antigen by activating the label, which can be used to determine the concentration of analyte in the sample. As used herein, the expression “sandwich complex” means an antibody-antigen-antibody sandwich.

In one example of the sandwich immunoassay format, a test sample containing an antibody is contacted with an antigen, e.g., a protein that has been immobilized on a solid phase material thereby forming an antigen-antibody complex. Examples of solid phase materials include beads, particles, microparticles, and the like. The solid phase material containing the antigen-antibody complex is typically treated, for example, with a second antibody that has been labeled with a detectable moiety. The second antibody then becomes bound to the antibody of the sample that is bound to the antigen immobilized on the solid phase material. Then, after one or more washing steps to remove any unbound material, an indicator material, such as a chromogenic substance, is introduced to react with the detectable moiety to produce a detectable signal, e.g. a color change, generation of light. The detectable signal change is then detected, measured, and correlated to the amount of antibody present in the test sample. It should also be noted that various diluents and buffers are also required to optimize the operation of the microparticles, antigens, conjugates, and other components of the assay that participate in chemical reactions. It should be further noted that other types of sandwich assays can be utilized, such as, for example, where the first antibody is immobilized on the solid phase material.

A heterogeneous immunoassay to determine the concentration of an analyte present at a low concentration in a biological sample can be performed with the apparatus described in U.S. Pat. Nos. 5,795,784 and 5,856,194, in a sandwich immunoassay format, which employs microparticles as the solid phase material. These patents are incorporated herein by reference.

In the case of HIV antigens, such as, for example, HIV-1 p24 antigen, it is preferred that monoclonal antibodies be used to carry out the immunoassay described herein. For example, monoclonal antibodies 120A-270 and 115B-151 can be used as a component of a solid phase capture antibody and as a detection antibody conjugate, respectively, to develop an ultra-sensitive immunoassay for HIV-1 p24 antigen for use in commercially available automated immunoassay analyzers. These monoclonal antibodies are described in greater detail in U.S. Pat. No. 6,818,392, incorporated herein by reference. Monoclonal antibodies are typically selected on the basis of their high binding affinities (e.g., greater than 5×10⁹ liters/mole), compatibility between components for sandwich assays, and detection of all subtypes of the antigen tested. The monoclonal antibodies for the HIV-1 p24 antigen mentioned previously can be used to determine all subtypes of HIV-1 p24 antigen and HIV-2 p26 antigen.

Determination of the presence and amount of an analyte in a biological sample can be determined by a competitive diagnostic assay. Small molecule, competitive diagnostic assays usually require a labeled component that can compete with the analyte for available antibody sites. The labeled component is typically referred to as a tracer. Examples of the labeled component include radioactive tracers, fluorescent tracers, chemiluminescent tracers, and enzyme tracers. Typically, the labeled component consists of the analyte or an analogue of the analyte coupled to a label.

The probability that a particular reagent comprising a specific binding member for a given analyte and a labeled component will be useful in a sensitive assay for the given analyte can be assessed by knowledge of the dose response curve. The dose response curve for an immunoassay is a plot of the ratio of the response in the presence of the subject analyte to the response in the absence of the subject analyte as a function of the concentration of the subject analyte. The dose response curve for a given immunoassay is unique for each reagent comprising a specific binding member for a given analyte and a tracer and is modulated by the competition between the tracer and the analyte for sites on the specific binding member for the analyte.

Prior to carrying out an immunoassay for the subject antigens, the method described herein utilizes a processing technique to prepare biological samples for use in a commercially available automated immunoassay analyzer. Such a processing technique can be carried out with a KingFisher™ mL magnetic particle processor or a KingFisher™ magnetic particle processor, both of which are commercially available from Thermo Fisher Scientific, Inc., Waltham, Mass.

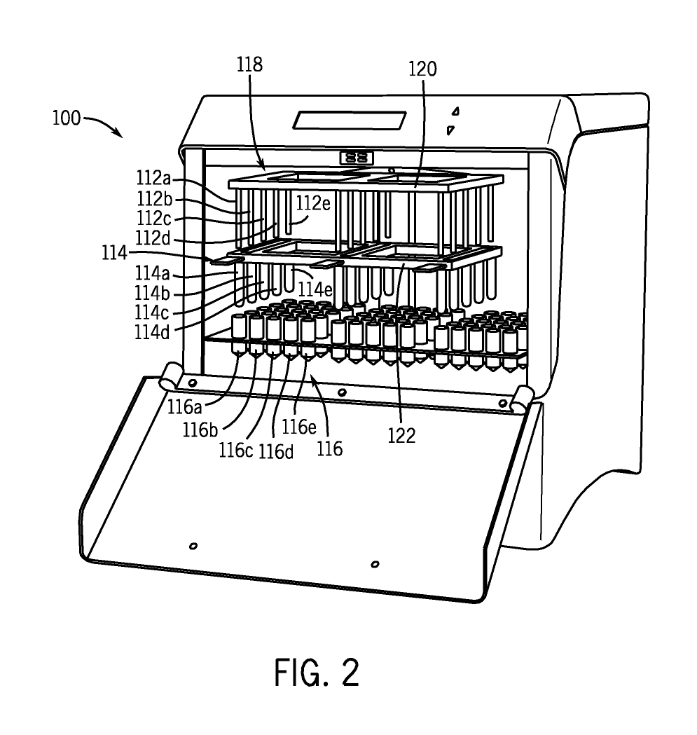

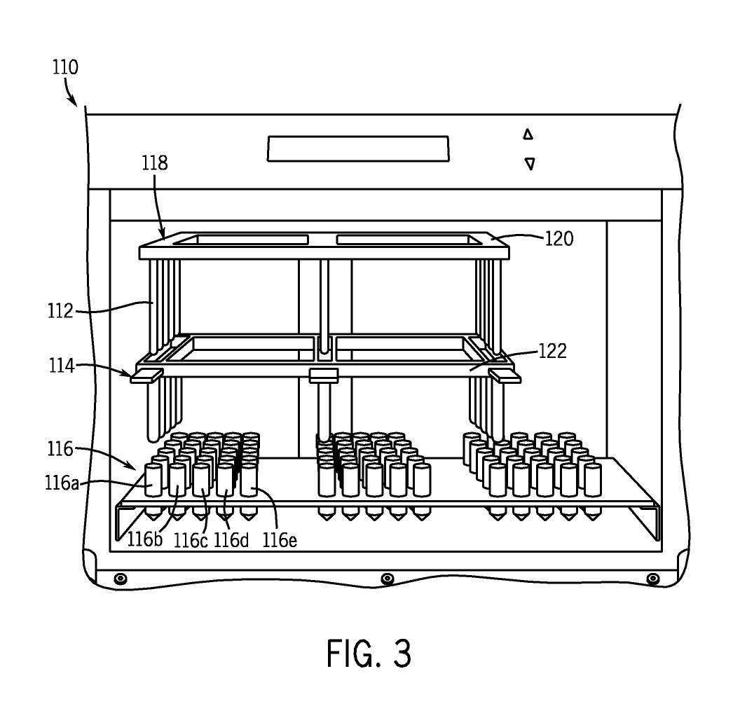

Referring now to FIGS. 2 and 3, a KingFisher™ mL magnetic particle processor 110 can be used for automated transfer and processing of magnetic particles in tubes of a tube strip. In the description that follows, the tubes of the tube embodiment will be used to illustrate the concentrating technique. The principle of the KingFisher™ mL magnetic particle processor 110 is based on the use of (a) magnetic rods 112a, 112b, 112c, 112d, and 112e that can be covered with disposable tip combs 114 and (b) tube strips 116. A tip comb 114 comprises a strip of non-magnetic material that joins a plurality of sheaths 114a, 114b, 114c, 114d, and 114e made of non-magnetic material for covering magnetic rods. A tube strip 116 is a plurality of tubes 116a, 116b, 116c, 116d, and 116e arranged in a row. The KingFisher™ mL magnetic particle processor 110 is capable of functioning without any aspiration and/or dispensing devices. The KingFisher™ mL magnetic particle processor 110 is designed for a maximum of fifteen (15) tube strips 116, which are compatible with the tip comb 114. The tube strip(s) 116 is (are) maintained stationary and the only movable assembly is a processing head 118 along with the tip combs 114 and magnetic rods 112a, 112b, 112c, 112d, and 112e associated therewith. The processing head 118 comprises two vertically moving platforms 120, 122. One platform 120 is needed for the magnetic rods 112a, 112b, 112c, 112d, and 112e, and the other platform 122 is needed for the tip combs 114. A tray 124 contains 15 separate tube strips 116 and a single sample processing typically uses one tube strip 116 containing five tubes 116a, 116b, 116c, 116d, and 116e. One tip comb 114 containing five tips 114a, 114b, 114c, 114d, and 114e is used for processing five samples at one time.

Before starting the magnetic particle processing via a keypad (not shown) and a display (not shown), the samples and reagents are dispensed into the tubes 116a, 116b, 116c, 116d, and 116e and the tip comb(s) 114 is (are) loaded into its (their) slot(s). The tube strip(s) 116 is (are) placed into the removable tray in the correct position and the tray is pushed into the end position. During the operation, the front and top lids can be closed or open. Closed lids protect the processing against environmental contamination. The KingFisher™ mL magnetic particle processor is described in detail in KingFisher™ mL User manual, Revision No. 1.0, February 2002, Catalog No. 1508260, incorporated herein by reference.

The KingFisher™ magnetic particle processor is designed for the automated transfer and processing of magnetic particles in volumes of liquids suitable for micro-wells. This is in contrast to the KingFisher™ mL magnetic particle processor, which employs greater volumes of liquids. The KingFisher™ magnetic particle processor is described in detail in KingFisher™ Micro-well User Manual, Revision No. 1.0, 1999-04-09, Catalog No. 1507730, incorporated herein by reference.

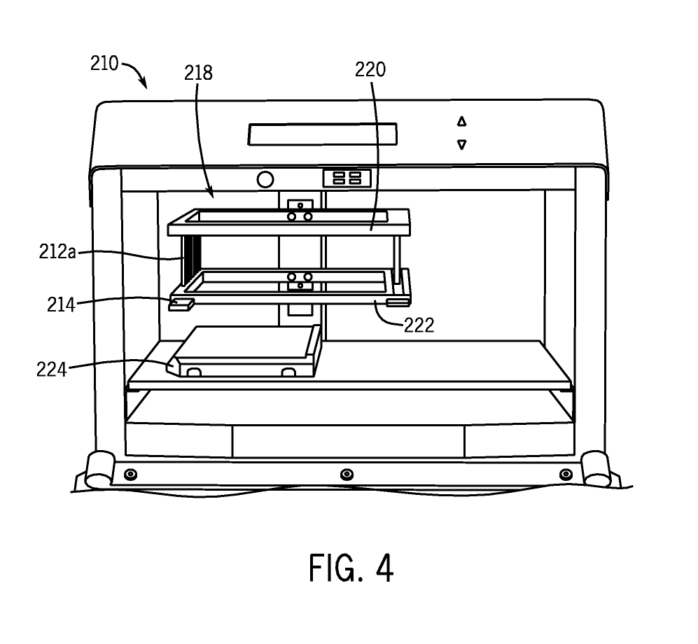

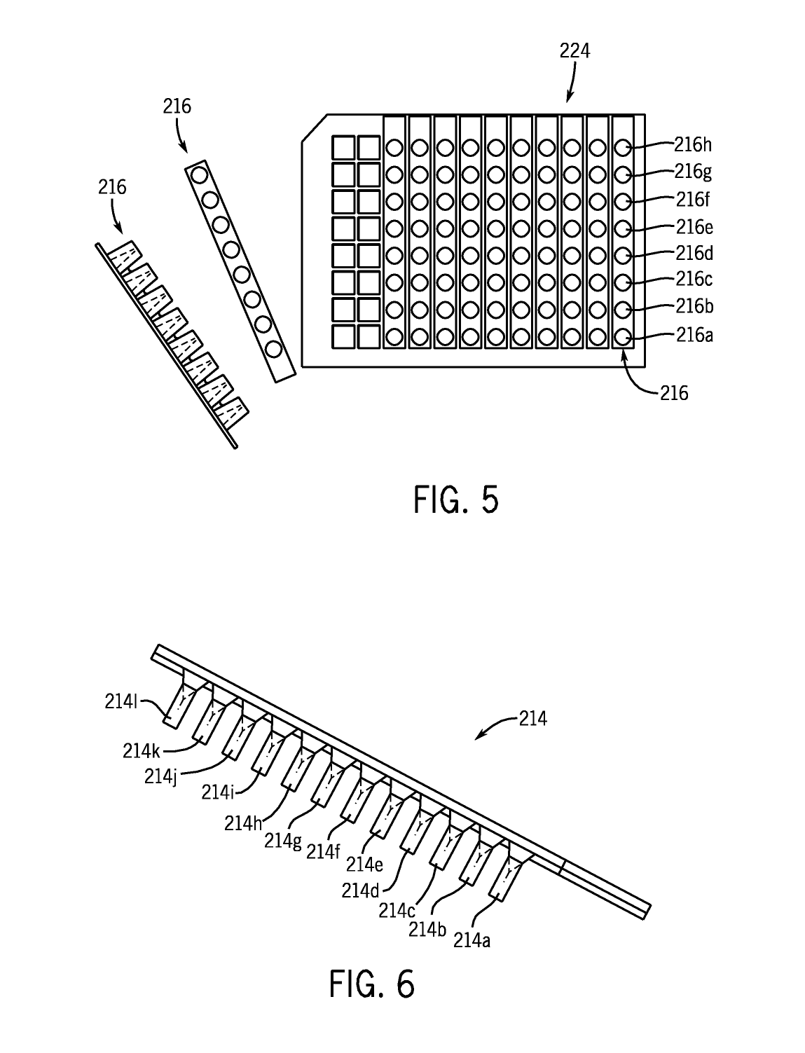

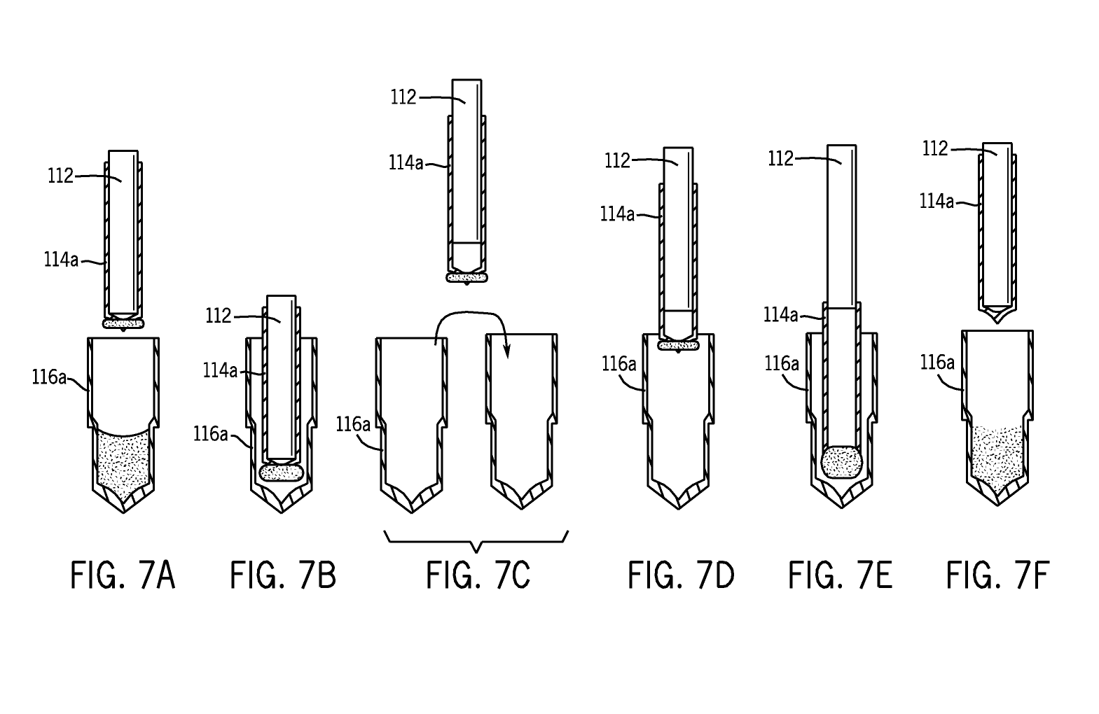

Referring now to FIGS. 4, 5, and 6, a KingFisher™ magnetic particle processor 210 can be used for automated transfer and processing of magnetic particles in wells of a micro-well plate. The principle of the KingFisher™ magnetic particle processor 210 is based on the use of magnetic rods 212a, 212b, 212c, 212d, 212e, 212f, 212g, 212h that can be covered with disposable tip combs 214 and well strips 216. Only the magnetic rod 212a is shown. The other magnetic rods are hidden by the magnetic rod 212a. A tip comb 214 comprises a strip of non-magnetic material that joins a plurality of sheaths made of non-magnetic material for covering magnetic rods. A well strip 216 is a plurality of micro-wells arranged in a row. The KingFisher™ magnetic particle processor 210 is capable of functioning without any aspiration and/or dispensing devices. The KingFisher™ magnetic particle processor 210 is designed for a maximum of ninety-six (96) micro-wells, which are compatible with the tip comb 214. The micro-wells are maintained stationary and the only movable assembly is a processing head 218 along with the tip combs 214 and magnetic rods 212 associated therewith. The processing head 218 comprises two vertically moving platforms 220, 222. One platform 220 is needed for the magnetic rods 212 and the other platform 222 is needed for the tip combs 214. A tray 224 contains one micro-well plate and a single sample processing typically uses one well strip 216 containing eight micro-wells 216a, 216b, 216c, 216d, 216e, 216f, 216g, and 216h. One tip comb 214 containing twelve tips 214a, 214b, 214c, 214d, 214e, 214f, 214g, 214h, 214i, 214j, 214k, and 214l is used for processing twelve samples at one time.

Before starting the magnetic particle processing via a keypad (not shown) and a display (not shown), the samples and reagents are dispensed into the micro-wells 216a, 216b, 216c, 216d, 216e, 216f, 216g, and 216h and the tip comb(s) 214 is (are) loaded into its (their) slot(s). The well strip(s) 216 is (are) placed into the removable tray in the correct position and the tray is pushed into the end position. During the operation, the front and top lids can be closed or open. Closed lids protect the processing against environmental contamination.

Regardless of which of the aforementioned KingFisher™ instrument is being used, the operating principle employed is inverse magnetic particle processing technology, commonly referred to as MPP. Rather than moving the liquids from one well to another, the magnetic particles are moved from the tube 116a (or from the micro-well 216a) to the tube 116b (or to the micro-well 216b), at least one tubes (micro-wells) containing specific reagent(s). This principle stands in contrast to the external magnet method, i.e., the type of separation used in the apparatus shown in U.S. Pat. Nos. 5,795,784 and 5,856,194. According to inverse magnetic particle processing technology, magnetic particles are transferred with the aid of magnetic rods covered with disposable, specially designed plastic tip combs.

Working with magnetic particles can be divided into five separate process steps:
Collecting particles: in this step, magnetic particles are collected from the well or tube specified.
Binding particles: In this step, material is collected onto the magnetic particles from the reagent in a specific well or tube.
Mixing particles: In this step, the reagent and particles (if inserted), are mixed with the plastic tip in a specific well or tube.
Releasing particles: In this step, the collected material is released from the surfaces of the magnetic particles into a specific well or tube.
Washing particles: In this step, the magnetic particles are washed in a specific well or tube.
Transferring particles: In this step, the magnetic particles are moved from one well or tube to another.
   During the collection of the magnetic particles, the magnetic rod is fully inside the tip. The magnetic rods together with the tip comb(s) move slowly up and down in the tubes and the magnetic particles are collected onto the wall of the tips. The magnetic rods together with the tip comb(s), having collected the magnetic particles, can be lifted out of the tubes and transferred into the next tubes. After collection of the magnetic particles, the magnetic rods together with the tip comb(s) are lifted from the tubes, the tip comb(s) is (are) lowered into the next tubes containing a reagent, the magnetic rods are lifted from the tip comb(s). Magnetic particles are released by moving the tip comb(s) remaining in the reagent up and down several times at considerably high speed until all the particles have been mixed with the substance in the next reaction tube. Washing the magnetic particles is a frequent and an important processing phase. Washing is a combination of the release and collection processes in a tube filled with washing solution. To maximize washing efficiency, the magnetic rods together with the tip comb(s) are designed to minimize liquid-carrying properties. To keep the magnetic particle suspension evenly mixed in long-running reactions, the tip comb(s) can be moved up and down from time to time. The volume of the first tube can be larger than the volume of the next tube and this is used for concentration purposes.

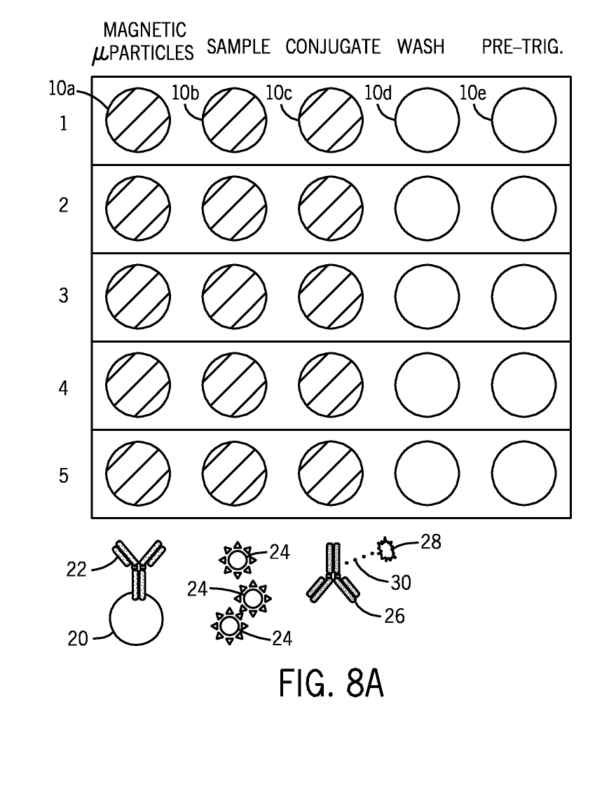

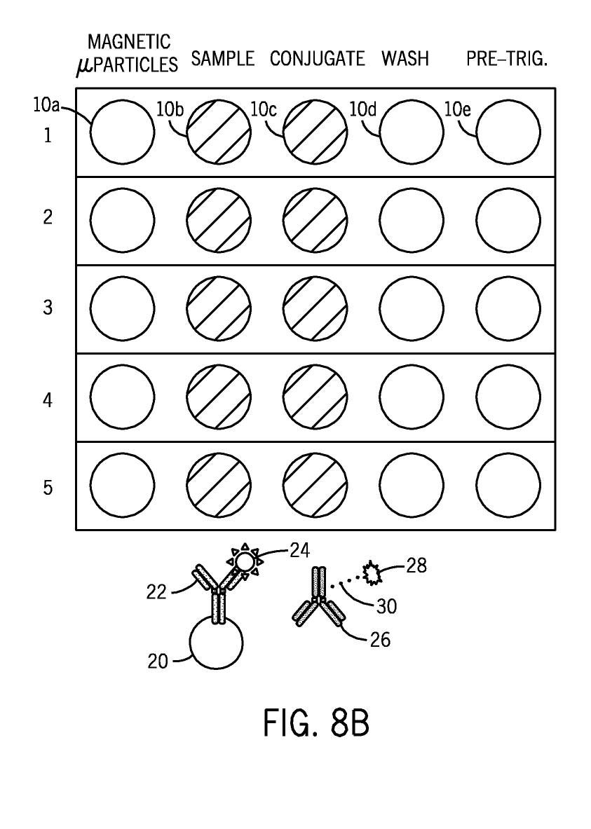

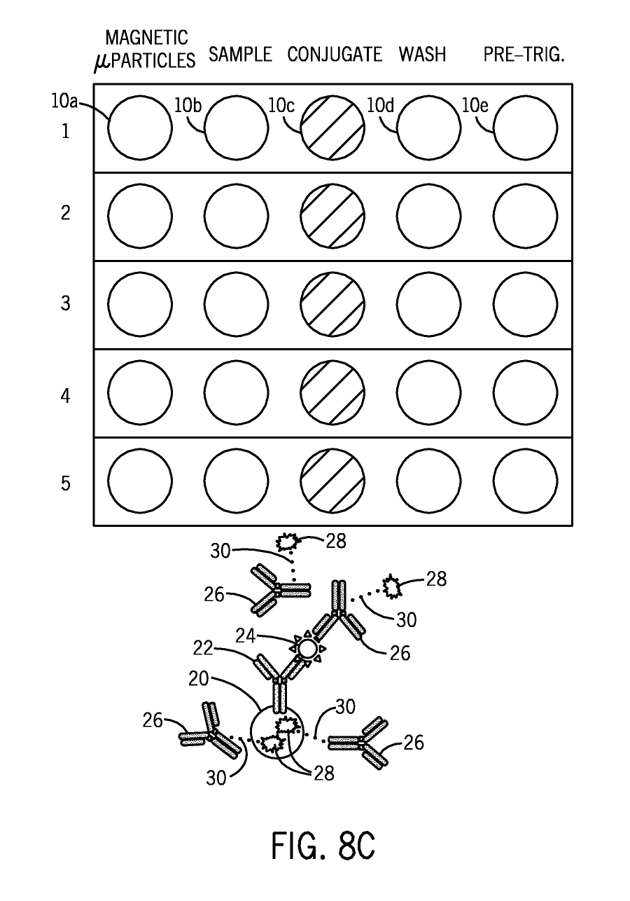

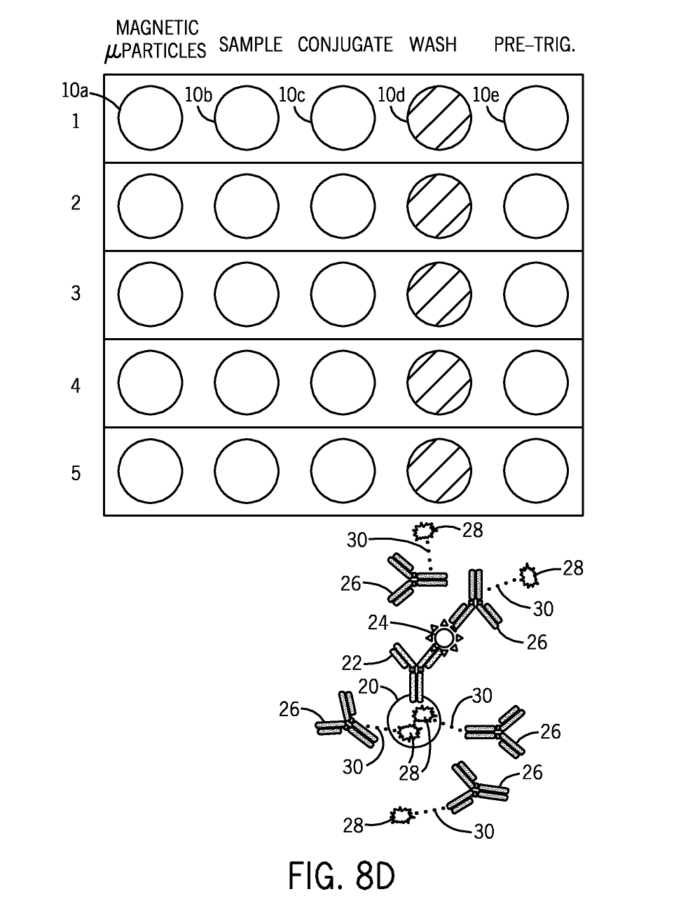

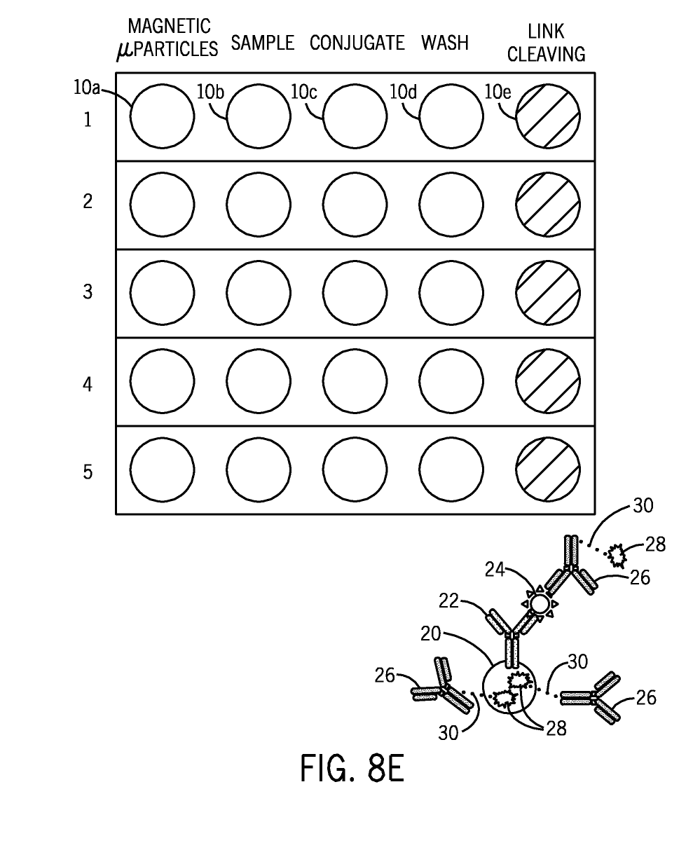

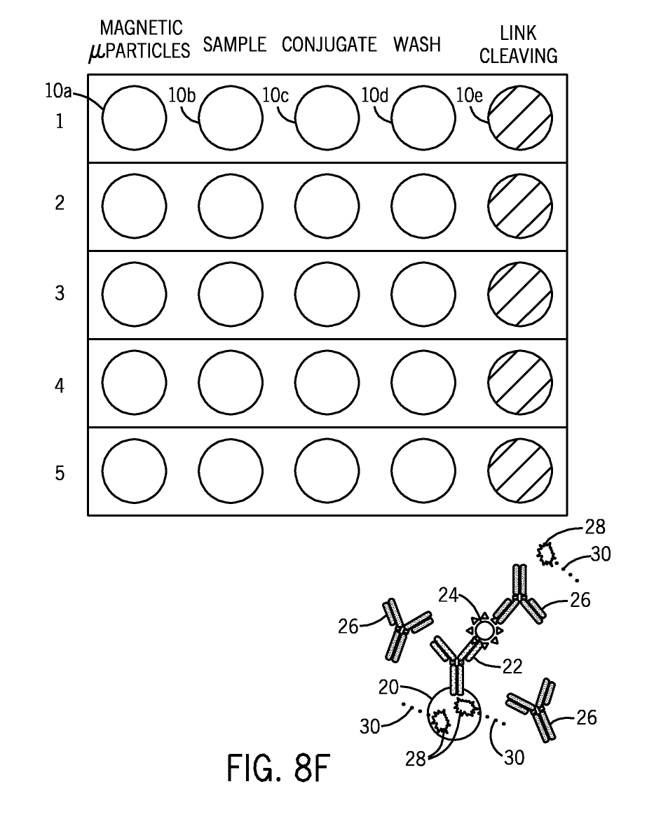

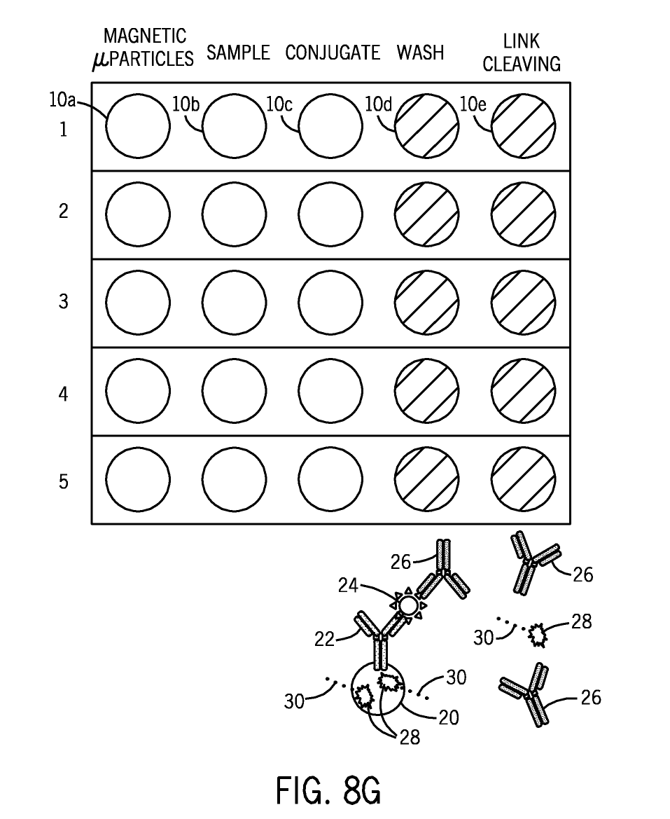

FIGS. 8A, 8B, 8C, 8D, 8E, 8F, and 8G illustrate the sequence of steps employed in collecting, transferring, and releasing magnetic particles from tubes in a KingFisher™ mL magnetic particle processor according to the method described herein. In FIGS. 8A, 8B, 8C, 8D, 8E, 8F, and 8G, there are five rows of containers, i.e., tubes, with five containers, i.e., tubes, in each row. The tubes in which operations for a given process step are being carried out are designated by hatch lines. Below the array of 25 tubes (5 rows×5 tubes/row) are schematic representations of (a) a first conjugate, (b) a sample, and (c) a second conjugate undergoing given operations for a given process step.

FIG. 8A shows tubes 10a, 10b, 10c, 10d, and 10e in row 1 at the starting point of the immunoassay. The tubes in rows 2, 3, 4, and 5 are identical to those in row 1. The magnetic microparticle is designated by the reference numeral 20. The specific binding member attached to the magnetic microparticle is designated by the reference numeral 22. The conjugate containing the magnetic microparticle 20 and the specific binding member 22 attached to the magnetic microparticle is referred to as the first conjugate. The analyte in the sample is represented by the reference numeral 24. The specific binding member attached to the label is designated by the reference numeral 26. The label itself is designated by the reference numeral 28. The conjugate containing the specific binding member 26 and the label 28 is referred to as the second conjugate. In FIGS. 8A, 8B, 8C, 8D, 8E, and 8F, in the first conjugate, the specific binding member 22 is covalently bonded to the magnetic microparticle 20. However, it is not required that the specific binding member 22 be covalently bonded to the magnetic microparticle 20. In an alternative embodiment, the specific binding member 22 can be attached to the magnetic microparticle 20 by means of van der Waals force. In FIGS. 8A, 8B, 8C, 8D, 8E, 8F, and 8G, the cleavable linking agent is designated by the reference numeral 30 and illustrated as a series of dots.

As shown in FIG. 8A, at the beginning of the immunoassay, a first conjugate is introduced into the tube(s) 10a, a sample is introduced into the tube(s) 10b, and a second conjugate is introduced into the tube(s) 10c. FIG. 8B shows that the first conjugate is being mixed with the sample in the tube(s) 10b. The specific binding member 22 of the first conjugate binds to the analyte 24 in the sample. FIG. 8C shows that the reaction product in the tube(s) 10b in FIG. 8B has been transferred to the tube(s) 10c containing the second conjugate, whereupon the specific binding member 26 of the second conjugate binds to the analyte 24 that is specifically bound to the specific binding member 22 of the first conjugate. In FIG. 8D, the complex formed in the reaction shown in FIG. 8C is washed in the tube(s) 10d, in order to remove the second conjugate that is not bound to the analyte 24. FIG. 8E shows the microparticle 20 with the specifically bound label 28 and the non-specifically bound label 28, after the reaction mixture is transferred into the final tube(s) 10e. FIG. 8F shows the effect of the cleaving step of the linking agent, whereby the label 28 is detached from the specific binding member 26 of the second conjugate in the tube(s) 10e. In FIG. 8G, the magnetic microparticle 20 is removed from the reaction mixture to the tube(s) 10d, whereupon the signal can be measured in the tube(s) 10e in order to determine the concentration of analyte 24 in the sample. In the step shown in FIG. 8G, the label 28 that is non-specifically bound to the magnetic microparticle 20 also remains bound to the magnetic microparticle 20. In addition, any conjugate comprising the second specific binding member 26 and the label 28 that is non-specifically bound to the magnetic microparticle 20 also remains bound to the magnetic microparticle. The magnet removes the magnetic microparticles 20 and all other entities bound to the magnetic microparticles 20, including the label 28 that is non-specifically bound to the magnetic microparticles 20. In the step shown in FIG. 8G, the signal is measured in high pH environment containing hydrogen peroxide.

With respect to non-specific binding, the label can non-specifically bind to magnetic microparticles; furthermore, the specific binding member can non-specifically bind to magnetic microparticles.

The removal of a chemiluminescent label, e.g., acridinium, that is non-specifically bound to magnetic microparticles has not been an option for chemiluminescent instrument platforms, e.g., ARCHITECT®, PRISM®, IMX®, AXSYM® instruments. Prior to the development of the conjugate and the method described herein, there has not been a mechanism available to separate the solid phase, with any non-specifically bound chemiluminescent label, e.g., acridinium, from the reaction mixture prior to the sequence of the trigger step, the read step, and the quantitation step, in these instruments.

The use of a conjugate having a cleavable linking agent, with a Kingfisher™ magnetic particle processor or a Kingfisher™ mL magnetic particle processor, allows only the label specifically bound to the captured analyte to contribute to a signal. The Non-specifically bound label attached to the solid phase, e.g., magnetic microparticles, would be removed from the elution well and eliminated as a source of a non-specific signal, thereby improving the sensitivity of the assay.

The method described herein provides an opportunity to evaluate the use of increased concentrations of labeled conjugates, or to evaluate the incorporation of higher ratios of label into conjugates when preparing conjugates comprising a specific binding member attached to a label.

Increased concentrations of labeled conjugates would accelerate reaction kinetics, and the incorporation of higher ratios of labels into conjugates would increase the amount of label present for the detections system. Either of these actions could improve sensitivity of the assay by increasing the signal based on the specifically bound analyte. Higher concentrations of the labeled conjugates or higher incorporation ratios of the label into the labeled conjugates would likely result in more non-specific binding of the labeled conjugates to the solid phase, e.g., magnetic microparticles. However, the non-specifically bound label would not increase the signal resulting from non-specifically bound conjugates, because such non-specifically bound label would be removed from the elution reaction mixture along with the solid phase, e.g., magnetic microparticles.

If acridinium is used as the label, the cleavable linking agent would need to be cleavable under conditions that do not trigger the acridinium prematurely, or modify the acridinium in such a way that would result in reduction, or even elimination, of its chemiluminescent properties.

Cleavable linking agents suitable for use with the reagents and immunoasssays described herein are set forth in TABLE 1.

TABLE 1

| | | Moieties with | |
|---|---|---|---|
| | | which linking | |
| | | agent will form | |
| Cleavable linking agent | Reactive groups | chemical bond | Cleaving agent |
| 3,3′-dithiobis[succinimydyl | NHS ester | Amino groups | Reducing agents, |
| propionate] | (homobifunctional) | | e.g., thiol group. |
| | | | The linker center, |
| | | | S═S, can be |
| | | | cleaved by a |
| | | | molecule that |
| | | | contains a thiol |
| | | | group. |
| 3-[(2- | amine group and | NHS esters/sulfo- | Reducing agents, |
| aminoethyl)dithio]propionic | carboxyl group | NHS esters and | e.g., thiol group. |
| acide•HCl | | amines/hydrazides | The linker center, |
| | | via EDC activation | S═S, can be |
| | | | cleaved by a |
| | | | molecule that |
| | | | contains a thiol |
| | | | group. |
| 1,4 bis-maleimydyl-2,3- | maleimide group | Sulfhydryl groups | Sodium meta- |
| dihydoxybutane | (homobifunctional) | | periodate |
| disuccinimydyl tartrate | NHS ester | Amino groups | Sodium meta- |
| | (homobifunctional) | | periodate |
| ethylene glycol bis | NHS esters | Amino groups | Hydroxylamine for |
| [sulfosuccinimydylsuccinate] | (homobifunctional) | | 3 to 6 hours at |
| | | | 37° C. at pH 8.5 |

Source: Pierce Catalog 2005/2006, incorporated herein by reference.

The cleavable linking agents suitable for use herein are insensitive to pH of the reaction mixture.

Techniques for preparing the first conjugate and the second conjugate are well-known to those having ordinary skill in the art. In order to prepare the first conjugate, the magnetic microparticle, which has a polymeric coating thereon, can be bound to the specific binding member in one of two ways. If the polymeric coating has reactive groups, the magnetic microparticle can be covalently bonded to the specific binding member. If the polymeric coating does not have reactive groups or if it has reactive groups that will not react with the specific binding member, the magnetic microparticle can be attached to the specific binding member by van der Waals force. The first conjugate suitable for use herein can be manufactured by Invitrogen Corporation, under the trademark Dynal®.

In order to prepare the second conjugate, one reactive group of the cleavable linking agent forms a bond with a functional group of the specific binding member, and the other reactive group of the cleavable linking agent forms a bond with a functional group of the label. See Pierce Catalog 2005/2006, incorporated herein by reference, for references that teach one of ordinary skill in the art how to attach the cleavable linking agent to functional groups of chemical entities, such as, for example, specific binding members and labels.

Conjugates suitable for use herein can be manufactured by Invitrogen Corporation.

The conjugate described herein has several advantages relative to those of the prior art. By removal of the non-specifically bound label, noise is reduced and the signal-to-noise ratio is increased. A higher concentration of the conjugate, e.g., acridinium attached to a specific binding member, can be added to the reaction mixture. A higher concentration of label, e.g., acridinium, can be incorporated into the conjugate.

Care must be taken in selection of the linking agent that the cleavage process must not adversely affect the label, e.g., acridinium. Care must be taken so that the range of pH in the presence of hydrogen peroxide does not exceed 8.0.

Various modifications and alterations of this invention will become apparent to those skilled in the art without departing from the scope and spirit of this invention, and it should be understood that this invention is not to be unduly limited to the illustrative embodiments set forth herein.

What is claimed is:

1. A conjugate comprising a specific binding member, a label, and a cleavable linking agent, wherein the specific binding member and the label are joined by the cleavable linking agent.

2. The conjugate of claim 1, wherein the cleavable linking agent is capable of bonding to the label.

3. The conjugate of claim 1, wherein the cleavable linking agent is capable of bonding to a specific binding member.

4. The conjugate of claim 3, wherein the specific binding member is an antobody.

5. The conjugate of claim 1, wherein the label is a chemiluminescent label.

6. The conjugate of claim 5, wherein the chemiluminescent label is acridinium.

7. The conjugate of claim 1, wherein the cleavable linking agent is selected from the group consisting of 3,3′-dithiobis[succinimydyl propionate], 3-[(2-aminoethyl)dithio]propionic acid.HCl, 1,4 bis-maleimydyl-2,3-dihydoxybutane, disuccinimydyl tartrate, and ethylene glycol bis[sulfosuccinimydylsuccinate].

8. An immunoassay comprising the steps of:
(a) providing a biological sample suspected of containing an analyte;
(b) providing a first conjugate comprising a solid phase material attached to a specific binding member specific for the analyte;
(c) providing a second conjugate comprising a specific binding member specific for the analyte, a label, and a cleavable linking agent, wherein the specific binding member specific for the analyte and the label are joined by the cleavable linking agent;
(d) mixing (a) the biological sample, (b) the first conjugate, and (c) the second conjugate in a container to form a reaction mixture;
(e) cleaving the label from the second conjugate;
(f) removing the label non-specifically bound to the solid phase material;
(g) measuring the signal generated by the label; and
(h) determining the concentration of analyte in the sample.

9. The method of claim 8, wherein the cleavable linking agent is capable of bonding to the label.

10. The method of claim 8, wherein the cleavable linking agent is capable of bonding to a specific binding member.

11. The method of claim 10, wherein the specific binding member of the first conjugate is an antibody and the specific binding member of the second conjugate is an antibody.

12. The method of claim 8, wherein the label is a chemiluminescent label.

13. The method of claim 12, wherein the chemiluminescent label is acridinium.

14. The method of claim 8, wherein the cleavable linking agent is selected from the group consisting of 3,3′-dithiobis[succinimydyl propionate], 3-[(2-aminoethyl)dithio]propionic acid.HCl, 1,4 bis-maleimydyl-2,3-dihydoxybutane, disuccinimydyl tartrate, and ethylene glycol bis[sulfosuccinimydylsuccinate].

15. An immunoassay comprising the steps of:
(a) providing a biological sample suspected of containing an analyte;
(b) providing a first conjugate comprising a solid phase material attached to a specific binding member specific for the analyte;
(c) providing a second conjugate comprising a specific binding member comprising the analyte, a label, and a cleavable linking agent, wherein the analyte and the label are joined by the cleavable linking agent;
(d) mixing (a) the biological sample, (b) the first conjugate, and (c) the second conjugate in a container to form a reaction mixture;
(e) cleaving the label from the second conjugate;
(f) removing the label non-specifically bound to the solid phase material;
(g) measuring the signal generated by the label; and
(h) determining the concentration of analyte in the sample.

16. The method of claim 15, wherein the cleavable linking agent is capable of bonding to the label.

17. The method of claim 15, wherein the cleavable linking agent is capable of bonding to a specific binding member.

18. The method of claim 17, wherein the specific binding member is an antibody.

19. The method of claim 15, wherein the label is a chemiluminescent label.

20. The method of claim 19, wherein the chemiluminescent label is acridinium.

21. The method of claim 15, wherein the cleavable linking agent is selected from the group consisting of 3,3′-dithiobis[succinimydyl propionate], 3-[(2-aminoethyl)dithio]propionic acid.HCl, 1,4 bis-maleimydyl-2,3-dihydoxybutane, disuccinimydyl tartrate, and ethylene glycol bis[sulfosuccinimydylsuccinate].

22. A kit comprising a conjugate, the conjugate comprising a specific binding member, a label, and a cleavable linking agent, wherein the specific binding member and the label are joined by the cleavable linking agent.

23. The kit of claim 22, wherein the cleavable linking agent is capable of bonding to the label.

24. The kit of claim 22, wherein the cleavable linking agent is capable of bonding to a specific binding member.

25. The conjugate of claim 24, wherein the specific binding member is an antobody.

26. The kit of claim 22, wherein the label is a chemiluminescent label.

27. The kit of claim 26, wherein the chemiluminescent label is acridinium.

28. The kit of claim 1, wherein the cleavable linking agent is selected from the group consisting of 3,3′-dithiobis[succinimydyl propionate], 3-[(2-aminoethyl)dithio]propionic acid.HCl, 1,4 bis-maleimydyl-2,3-dihydoxybutane, disuccinimydyl tartrate, and ethylene glycol bis[sulfosuccinimydylsuccinate].
